(19) United States
(12) Patent Application Publication
(10) Pub. No.: US 2026/0224815 A1
(43) Pub. Date: Aug. 6, 2026

(54) DEVICES AND METHODS FOR A VARIABLY LOCKING SYRINGE ASSEMBLY

(71) Applicant: Femasys Inc., Suwanee, GA (US)

(72) Inventors: Kathy Lee-Sepsick, Suwanee, GA (US); Jeremy Sipos, Alpharetta, GA (US)

(21) Appl. No.: 19/155,065

(22) PCT Filed: Jan. 27, 2024
(86) PCT No.: PCT/US2024/013265

§ 371 Date: Aug. 8, 2025

Related U.S. Application Data
(60) Provisional application No. 63/444,605, filed on Feb. 10, 2023.

(51) Int. Cl. A61M 5/315; A61M 5/31; A61M 25/10

(52) U.S. Cl. CPC A61M 5/31501; A61M 5/3134; A61M 25/10182

(57) ABSTRACT

Disclosed herein are exemplary variably lockable syringe assemblies, methods of making and using the same. Such variably locking syringe assemblies are useful in many applications.

### RELATED APPLICATIONS

The present disclosure is a PCT application claiming the priority to and benefit of U.S. Provisional Patent Application Ser. No. 63/444,605, filed Feb. 10, 2023, which is herein incorporated in its entirety. Any and all applications for which a foreign or domestic priority claim is identified in the Application Data Sheet as filed with the present application are hereby incorporated by reference.

### TECHNICAL FIELD

The present disclosure generally discloses syringe assemblies and related devices and methods. The present disclosure relates to syringe assemblies wherein the plunger is lockable at a position within the syringe barrel and to devices and methods related to the syringe assemblies.

### BACKGROUND OF THE INVENTION

Syringes are used in many medical and non-medical applications, for example, to withdraw blood, inject medicines, with other devices, and to aid in biopsy and bone-marrow transplant procedures. A medical device syringe or syringe assembly generally includes a barrel and a plunger that is slideable within the barrel and which sealingly fits within the barrel. The plunger commonly is provided with an accessible finger grip at its proximal end for pushing and pulling the plunger into and out of the barrel, and the barrel itself may have opposing finger grips at its proximal end. Medical syringes have been made in a variety of sizes and configurations, and are well-known in the medical and industrial fields. Syringe barrels often contain volume markings, and often, the barrel is made of a translucent plastic material so that the position of the seal at the end of the plunger can be viewed through the barrel walls.

To control the amount of material moved into or out of a syringe barrel's interior, a stopcock may be attached to the distal end of a syringe barrel or is fluidly in-line with the inlet/outlet port of a syringe. Stopcock valve assemblies have long been used routinely in a variety of medical procedures and operations such as, for example, metering the infusion of fluids into the bodies of persons under medical care. An example of a stopcock valve assembly may include at least a three-way type valve wherein the valve body has three or more radially-arranged ports, and an interior directional control member is rotatably arranged within the valve body for selectively closing one or more ports while opening communication between at least two other ports. Use of a stopcock to control syringe material delivery introduces opportunities for contamination and requires an operator to consider the positioning of the stopcock valve handles and the amount of material within the syringe barrel.

What is needed are syringe assemblies that are easily and variably lockable so that the amount of material provided by the syringe is controlled, and in which the plunger is maintained in a stable position so that pressure from the material provided or from body tissues do not cause the plunger to move. Additionally, providing a syringe assembly that can maintain the plunger in a variably fixed position also helps prevent operator error.

### SUMMARY

The present disclosure provides methods for making and using devices comprising syringe assemblies that are easily and variably lockable so that the amount of a material provided by the syringe is controlled, and in which the plunger can be maintained in a stable position so that pressure from the material provided or from body tissues do not cause the plunger to move. Devices disclosed herein provide elements that allow the plunger to be stabilized or locked at one or many possible positions within the barrel, thus the syringe assembly is variably lockable. Devices disclosed herein may be locked at one position, unlocked and moved to a subsequent location and locked again. These steps can be repeated. Maintaining the plunger in a variably fixed position helps prevent operator error, such as by preventing the operator from accidentally retracting the plunger in a proximal direction or by providing too much material by pushing the plunger in a distal direction. In many procedures, while fluid is in or is controlled by the syringe, care should be taken that the plunger is not accidentally moved, and the presently disclosed devices are useful in such methods.

### FIGURES

The embodiments disclosed herein will become more fully apparent from the following description and appended claims, taken in conjunction with the accompanying drawings. These drawings depict only typical embodiments, which will be described with additional specificity and detail through use of the accompanying drawings. Alternative configurations of elements shown herein can be understood from the disclosure and figures herein.

FIG. 1 shows a drawing of an exemplary transparent syringe assembly disclosed herein wherein the plunger is removed from the barrel.

FIG. 2 shows a drawing of a front perspective view of an exemplary transparent syringe assembly disclosed herein.

FIG. 3 shows a drawing of a back perspective view of an exemplary transparent syringe assembly disclosed herein.

FIG. 4 shows a drawing of an exemplary transparent syringe assembly disclosed herein wherein the plunger is extended proximally from the barrel.

### DETAILED DESCRIPTION

Disclosed herein are syringe assembly devices and methods of making and using such devices. In an aspect, devices disclosed herein relate to a syringe assembly comprising at least a syringe barrel and a plunger. Disclosed herein are syringe assembly devices that are a syringe assemblies that are “lockable”, meaning that the plunger within the barrel can be maintained, and is stationary, at one of many possible longitudinal locations within the barrel. Once the syringe assembly operator positions the plunger in a locked position within the barrel, the plunger will remain at that position without further effort or activity by the operator. The locked plunger remains at the desired longitudinal position within the syringe barrel. When it is desired to move the plunger from the locked longitudinal position within the syringe barrel, the operator axially rotates (rotation around the longitudinal axis of the plunger, for example in a clockwise or counterclockwise direction) the plunger within the barrel until the plunger is in an unlocked position and is no longer lockable, and moves the plunger longitudinally in a distal or proximal direction as desired. When the plunger reaches the second or subsequent longitudinal position, the plunger is axially rotated to a locked position. The syringe assembly can be easily locked and released, and moved to another longitudinal position and relocked, which is what is meant by the device being variably lockable. Variably lockable means the syringe's ability to lock the plunger at one of multiple longitudinal locations throughout the barrel, meaning that the plunger can be positioned or repositioned in a different or the same longitudinal position; or the syringe assembly plunger can be unlocked or released by rotating the plunger in an axial direction, clockwise or counterclockwise, and moving the plunger from the locked position.

Variably locking syringe assemblies may be used in known medical applications, and can be used in applications where a flexible or variable response is needed. For example, consider an application that requires deploying a balloon that is attached to a syringe assembly. For some subjects, the balloon may be completely deployed, i.e., inflated, without causing pain or disruption to the subject. In this case, a disclosed syringe assembly would be used to fully inflate the balloon, i.e., provide air or fluid into the balloon, then the plunger would be locked in the desired longitudinal position in the barrel to maintain the balloon at its fully inflated state for the desired time needed, for example, for a medical procedure. Once the plunger is locked in place, with the balloon fully inflated, the operator no longer needs to interact with the syringe assembly. When the desired time is over and the balloon is to be deflated, the plunger is unlocked in the barrel and the plunger is moved longitudinally to deflate the balloon. For other patients, a fully inflated balloon is too large and/or provides too much distension, which may cause pain and/or disruption for the subject. Without the need to change to a smaller balloon, a variably locking syringe can be used to provide air or fluid into the balloon to inflate the balloon to a desired level of inflation that is satisfactory for the medical procedure for that subject. The plunger is then locked at the longitudinal position in the barrel that maintains the attached balloon at the desired level of inflation. Once the plunger is locked in place, with the balloon inflated to a less than fully deployed volume, the operator no longer needs to interact with the syringe assembly. When the desired time is over and the balloon is to be deflated, the plunger is unlocked in the barrel and the plunger is moved longitudinally to deflate the balloon. In an aspect, a differently sized variable locking syringe may be used in such procedures.

For example, variably locking syringe assemblies are also useful in medical applications where fluids, such as medications or injectable materials, are injected or provided to a subject, or removed from a subject, in a multi-step, measured, and/or time-controlled method. For example, variably lockable syringe assemblies may be useful for withdrawing fluids from within a subject. An operator can move the plunger in the barrel from a distal longitudinal position to a proximal longitudinal position to create a negative gauge pressure or vacuum inside the syringe barrel. The operator may then lock the plunger at a desired location within the syringe barrel to maintain the negative gauge pressure, facilitating withdrawal of fluid from within the patient. In a step-wise fashion, the plunger may be unlocked from the first position and moved longitudinally within the syringe barrel and be relocked in a different position to increase the pressure or vacuum, or reestablish the pressure or vacuum, or lessen the pressure or vacuum, or eliminate the pressure or vacuum. Alternatively, a fluid may be injected into a subject. For example, in a procedure that provides a known amount of a fluid multiple times to a subject may employ a variably locking syringe assembly. For example, in a procedure to alter a subject's face by providing a bulking material, such as hyaluronic acid, hydroxy apatite, or fat to one or more locations in the face, an operator can use a variably locking syringe assembly to reliably provide the same amount of the bulking material at each site injected, for example, down the length of a wrinkle. A needle attached to a variably locking syringe assembly is injected into one site of a wrinkle and the plunger is moved distally to inject a desired amount of bulking material at that site. The plunger is locked at that position, the needle is withdrawn from that site and reinjected into a second site. At the second site, the plunger is unlocked, the desired amount of bulking material is injected, the plunger is locked again, and the needle is withdrawn. The step may be repeated once or multiple times. The desired and known amount of material, e.g. fluid, air, gel, is reliably and reproducibly provided to the subject without errors due to the operator inability to maintain the plunger position during injection, after delivery and/or removal of the needle in one or more repeated steps. These examples of use are not intended to be limiting as to the disclosed devices and methods, as those of skill in the art can understand and perform procedures where a variably lockable syringe assembly can be employed.

Aspects of disclosed devices may be best understood by reference to the drawings, wherein like parts are designated by like numerals throughout. It will be readily understood that the components of disclosed devices as generally described and illustrated in the figures herein could be arranged and designed in a wide variety of different configurations. Thus, the following more detailed description of various embodiments, as represented in the figures, is not intended to limit the scope of the disclosure, but is merely representative of various aspects. In some cases, well-known structures, materials, or operations are not shown or described in detail. While the various aspects of the disclosed devices are presented in drawings, the drawings are not necessarily drawn to scale unless specifically indicated.

The phrases “connected to,” “coupled to,” and “in communication with” refer to any form of interaction between two or more entities, including but not limited to mechanical, electrical, magnetic, electromagnetic, fluid, and thermal interaction. Two components may be coupled to each other even though they are not in direct contact with each other. For example, two components may be coupled to each other through an intermediate component. The terms “abut” and “abutting” refer to items that are in direct physical contact with each other, although the items may not necessarily be attached.

The terms “distal” and “proximal” are given their ordinary meaning in the art. That is, the distal end of a medical device means the end of the device furthest from the operator during use. The proximal end refers to the opposite end, or the end nearest the operator during use. As specifically applied to a syringe assembly, the distal end of the syringe assembly refers to the end nearest the inlet/outlet port of the syringe barrel and the proximal end refers to the opposite end, the end nearest the head of the plunger. Further, it will be appreciated that the phrase “distal end” always refers to the end nearest the inlet/outlet port, even if the distal end is temporarily closer to the operator at one or more points in a procedure.

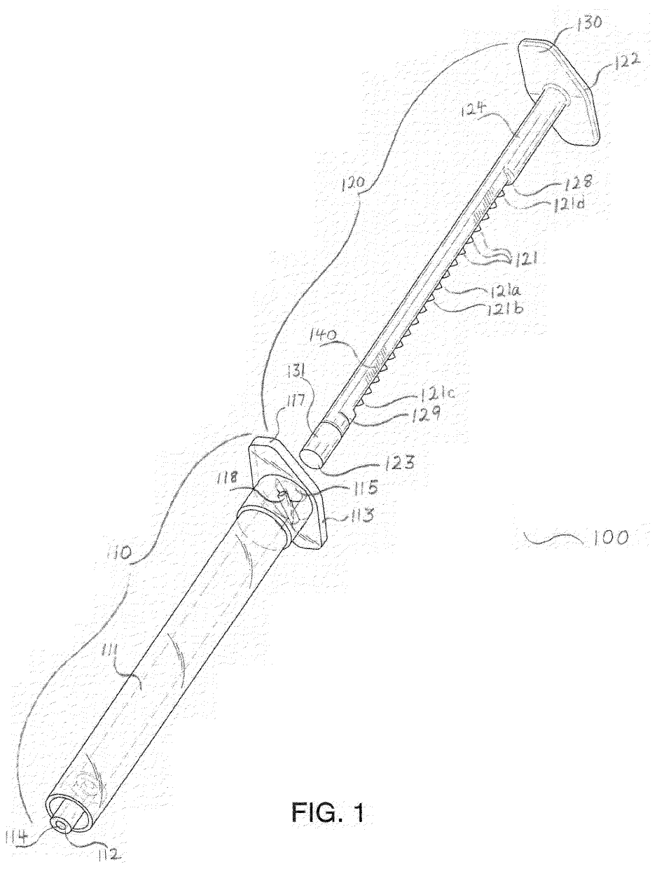

The disclosure may be further understood from the FIGs. provided herein. FIG. 1 shows an embodiment of a syringe assembly, syringe assembly 100. As shown in FIG. 1, syringe assembly 100 may comprise a barrel 110 and a plunger 120. Barrel 110 is a cylindrical tube comprising an interior lumen of the syringe assembly wherein plunger 120 can be pushed in (in a distal direction) or pulled out (in a proximal direction). Barrel 110 can be made of any suitable material, for example, polypropylene. Plunger 120 can be positioned within the interior of barrel 110, which can be pushed in (in a distal direction) or pulled (in a proximal direction). If pulled (in a proximal direction), a material such as a liquid, gel or gas will be drawn through inlet/outlet port 114 and inside interior lumen 111 of barrel 110. If pushed in (in a distal direction) by movement of plunger 120, the fluid or the gas will be expelled out inlet/outlet port 114. As shown in FIG. 1 and subsequent figures, barrel 110 is drawn as transparent so that the plunger is visible within interior lumen 111 of barrel 110. Aspects of the syringe assembly may comprise barrels and/or plungers that are not transparent. Further, barrel 110 may comprise markings or indicia within, on, affixed to or etched into the outer surface of barrel 110, such markings are not shown in the figures. Such markings or indicia may be horizontal lines and/or numbers, and for example, may be used to determine the volume of the material, fluid, liquid, gel or gas, within the barrel. Such markings or indicia on the barrel of a syringe assembly are known to those of skill in the art. Barrel 110 is hollow or substantially hollow. In an aspect, barrel 110 comprises an elongated cylindrical tube having lumen 111 extending therethrough.

Barrel 110 comprises a distal end 112 and a proximal end 113. Distal end 112 of barrel 110 may comprise distal inlet/outlet port 114 which is an opening in fluid connection with lumen 111. Barrel proximal end 113 may comprise proximal opening 115, throughwhich shaft 124 of plunger 120 traverses. Distal inlet/outlet port 114 is an opening or passage throughwhich material or fluids, such as liquids, gels and gases, are drawn into and/or pushed out of barrel 110. Distal end 112 of barrel 110 may be configured or shaped so that distal end 112 can mate or couple with an article, e.g., a catheter, medical device, tubing, a needle or other device, which can facilitate introduction or removal of fluids, attachment for transferring fluids, etc. For example, distal end 112 of barrel 110 may be shaped as a fitting, or to attach to a fitting, for example, shaped as a luer lock fitting, so that distal end 112 can mate with a complementary luer lock fitting on an article, e.g., catheter, tubing, medical device or needle to form a fluid connection between the interior of barrel 110 and the article. The luer lock fitting may be threaded. Many types of fittings, used for joining a syringe assembly to an article, are contemplated by the present disclosure, are known to those of skill in the art and may be used with disclosed syringe assemblies herein.

Proximal end 113 of the barrel 110 comprises proximal opening 115 through which plunger 120 extends and/or is inserted. Proximal end 113 of barrel 110 may comprise one or more barrel flange 117. One or more barrel flange 117 may extend outwardly from the barrel 110, as shown in the figures herein, and may be helpful in use of syringe assembly 100. The shape of one or more barrel flange 117 is not intended to be limited to only that shown in the figures herein and may be shaped differently, be curved, comprise ridges or indentations, be coated with a non-slip material or a coating, or other designs that aid in use of a disclosed syringe assembly.

Disposed within the interior of barrel 110 and proximate to proximal opening 115 is barrel key 118. Barrel key 118 may have any shape that allows barrel key 118 to fit between two plunger teeth 121, for example between tooth 121a and tooth 121b, and “lock” plunger 120 at a particular longitudinal position. For simplicity, only 3 teeth are identified as plunger teeth 121 in the figures, but it is to be understood that plunger teeth 121 comprises each and all of the plunger teeth shown in the figures. Barrel key 118 is shaped to interact with, and lock in a specific location, plunger 120. Barrel key 118 is shaped to interact with one or more plunger teeth 121 comprised by plunger 120. For example, teeth 121 may be beveled or chamfered so that when barrel key 118 is engaged with at least one of teeth 121 and edge 128 or edge 129, or two of teeth 121, plunger 120 is stabilized and held at that position within barrel 110, i.e., locked. For clarity of understanding, positioning barrel key 118 between two of any of teeth 121 or between a tooth and an edge, locks plunger 120 in a particular longitudinal position in barrel 110.

Plunger 120 has a proximal end 122 and a distal end 123, and is generally a solid shaft 124, that may be rounded, that moves longitudinally within lumen 111 of barrel 110. Optionally, proximal end 122 comprises plunger head 130, and positioned at distal end 123 is sealing feature 131. Sealing feature is an element that may form a seal so that material contained within barrel lumen 111 does not leak in a proximal direction, for example a gasket or a ring feature. As shown in FIG. 1, plunger 120, is removed from within barrel 110. Plunger head 130 may have any desired shape, for example, matching or corresponding to barrel flange 117, or may be different from barrel flange 117 and is not limited to the shape shown in the figures. Plunger head 130 provides a surface for touching or activation by an operator in moving plunger 120 distally or proximally. Optionally, plunger head 130 may have a larger diameter than the diameter of barrel 110, such as a shape that complements or engages with barrel flange 117, and sealing feature 131 may have a diameter such that sealing feature 131 forms a seal for shaft 124 within barrel lumen 111.

As shown in the figures, two structures are formed into longitudinal shaft 124 of plunger 120 and each structure is present on adjacent portions of longitudinal shaft 124. One structure is teeth section comprising teeth 121 and a proximal edge and a distal edge, and is partially shown in FIG. 1. Each one of teeth 121, is referred to herein singly as tooth 121, and can be indicated with an alphabetical symbol, as in tooth 121a or tooth 121b. As shown, teeth 121 are cut into shaft 124 so that the section of shaft 124 comprising teeth 121 has the same radius as shaft 124 and the diameter of shaft 124 is maintained so that plunger 120 moves easily within lumen 111. Optionally, plunger flange 130 may have a larger diameter than the diameter of barrel 110, such as a shape that complements or engages with barrel flange 117, and sealing feature 131 may have a diameter such that sealing feature 131 forms a seal in shaft 124.

A second structure of shaft 124 is flat section 140, which is adjacent to teeth 121, and is cut into shaft 124 and extends longitudinally along shaft 124 which may be at least the length of teeth 121 and may extend from edge 128 to edge 129. When plunger 120 is axially rotated around its longitudinal axis, so that flat section 140 faces barrel key 118, so that flat section 140 is adjacent to barrel key 118, plunger 120 can move easily in a proximal or distal direction within barrel 110 and cannot be locked. This is the released position. Looking at FIG. 1, if plunger 120 were inserted in a distal direction into barrel 110, flat section 140 would be facing barrel key 118 and plunger 120 would be in the released position.

To lock the syringe assembly, the following steps can be performed. Again looking at FIG. 1, for example, plunger 120 is inserted into lumen 111 of barrel 110, and flat section 140 is facing barrel key 118, with plunger 120 in the released position, then plunger 120 is moved distally longitudinally so that a portion of flat section 140 is at the desired longitudinal position, for example, the portion of flat section 140 located at tooth 121a is adjacent to barrel key 118. Plunger 120 is then axially rotated, for example approximately 90 degrees, so that tooth 121 a is positioned proximally of barrel key 118 and tooth 121 b is positioned distally of barrel key 118. Plunger 120 is now immobilized in a longitudinal direction and cannot move proximally or distally. This is the locked position. The locked position can be accomplished by positioning barrel key 118 between any two of teeth 121, or between the proximal-most tooth 121d and proximal edge 128, or distal-most tooth 121c and distal edge 129. Thus, syringe assembly 100 is variably lockable because barrel key 118 can be variably positioned between any two teeth or between the proximal-most tooth 121d and proximal edge 128, or distal-most tooth 121c and distal edge 129.

To unlock the syringe assembly and release plunger 120, plunger 120 is rotated, such as approximately 90 degrees in a direction opposite that used to lock plunger 120, to the released position so that flat edge 140 is facing barrel key 118, and then plunger 120 can move in a distal or proximal longitudinal direction as desired.

The size and dimensions of barrel 110 and plunger 120 may vary as desired, but teeth 121 and flat section 140 are included and shaped to provide syringe assembly 100 variable locking capability.

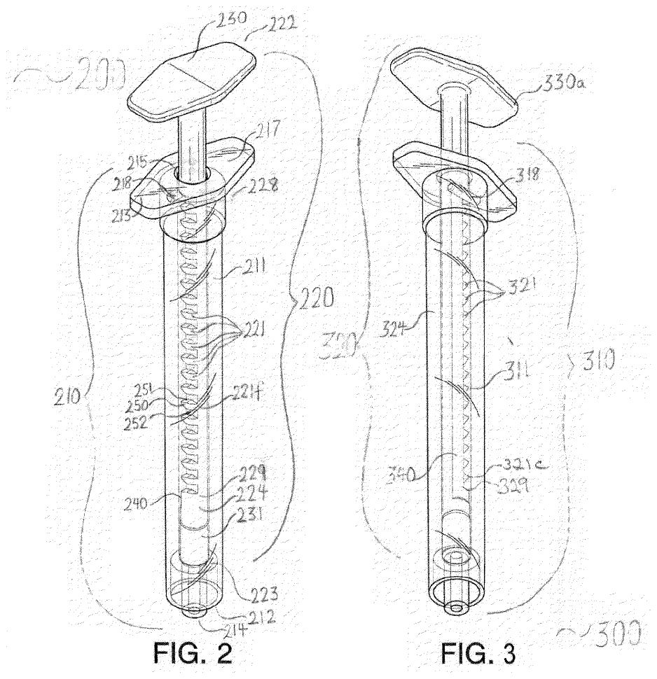

FIG. 2 shows a front side view of syringe assembly 200 with plunger 220 inserted into lumen 211 of barrel 210. Barrel 210 comprises a distal end 212 and a proximal end 213. Distal end 112 of barrel 210 comprises distal inlet/outlet port 214 which is an opening in fluid connection with lumen 211. Barrel proximal end 213 comprises proximal opening 215 and barrel flange 217, as described for FIG. 1. Distal inlet/outlet port 214 is an opening or passage throughwhich fluids, such as materials, e.g., liquids, gels and gases, are drawn into and pushed out of barrel 210. Distal end 212 of barrel 210 may be configured or shaped as described for FIG. 1. Proximal end 213 of barrel 210 comprises proximal opening 215 through which plunger 220 extends and/or is inserted.

Plunger 220 comprises shaft 224, has a proximal end 222 and a distal end 223, and can move longitudinally within lumen 211 of barrel 210. Optionally, proximal end 222 comprises plunger head 230, and positioned at distal end 223 is sealing feature 231. Plunger 220 comprises teeth 221 and flat section 240 (not visible).

As shown in FIG. 2, plunger 220 is rotated so as to be in the released position with flat section 240 facing barrel key 218. In FIG. 2, it can be seen that teeth 221 are formed in shaft 224 so that a tooth, for example, tooth 221f, is a protrusion with an open space above and below, protrusion 250 and space 251 and space 252. Individual teeth 221 are spaced apart a distance that may be slightly larger than the diameter of barrel key 218 so that barrel key 218 can be positioned between two of any of teeth 221 or can be positioned between proximal-most tooth and edge 228 or between distal-most tooth and edge 229. Barrel key 218 may have any desired shape and teeth 221 can be shaped accordingly to hold and maintain barrel key 218 therebetween, or between a tooth and an edge. As shown in FIG. 2, teeth 221 are shaped as chamfered teeth, but this is not to be seen as limiting, as teeth 221 can be any shape that allows for positioning barrel key 218 therebetween, or between a tooth and an edge.

FIG. 3 shows a back side view of syringe assembly 300 wherein flat section 340 is shown. Plunger 320 is shown inserted into lumen 311 of barrel 310 and is in the released position wherein flat section 340 is rotated to face barrel key 318. For example, to lock syringe assembly 300, plunger 320 is moved, if needed, in a proximal direction and, then rotated in a clockwise direction (or moving plunger head 330a towards the viewer) so that barrel key 318 is positioned between two of teeth 321, or between a tooth and an edge, for example between tooth 321c and edge 329. To unlock syringe assembly 300 and release plunger 320, plunger 320 is rotated in the reverse direction (counterclockwise or moving plunger head 330a away from the viewer to the position shown in FIG. 3) and flat section 340 is again facing barrel key 318. In this released or unlocked position, plunger 320 can move freely within lumen 311.

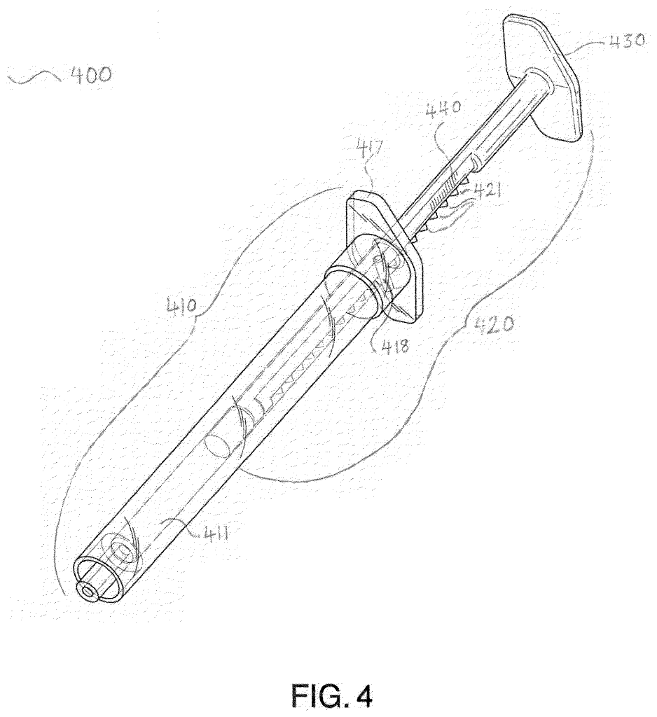

FIG. 4 shows a perspective view of a syringe assembly 400 disclosed herein. FIG. 4 shows plunger 420 positioned in lumen 411 of barrel 410 in a released position. Flat section 440 is facing barrel key 418 and plunger 420 can move freely longitudinally within lumen 411. In an aspect, positioning of barrel flange and plunger finger grip may provide a visual indicator to an operator of the position of the plunger and whether the plunger is in a locked position or an unlocked position. For example, as shown in FIG. 4, barrel flange 417 and finger grip 430 are shaped similarly and are aligned so that barrel flange 417 and finger grip 430 would correspond (or mirror) with each other. This aligned position is a visual key to the operator that the plunger is unlocked because flat section 440 is facing or adjacent to barrel key 418. When plunger 420 is axially rotated so that barrel flange 417 and finger grip 430 are not aligned, two of teeth 421 would then bracket barrel key 418. Barrel key 418 is then positioned between two of teeth 421 (or a tooth and an edge) and plunger 420 is immobilized in a longitudinal direction and cannot be moved distally or proximally. The unaligned position of the barrel flange and the finger grip indicates that the plunger is in a locked position. Axially rotating plunger 420 in the reverse direction, thereby realigning finger grip 430 and barrel flange 417, removes barrel key 418 from between two teeth 421 (or a tooth and an edge) and positions flat section 440 so that it faces barrel key 418, and releases plunger 420 to move freely in lumen 411 in a proximal or distal direction. It is to be understood that syringe assemblies disclosed herein are not required to have correspondingly shaped barrel flange and finger grip so as to indicate the position of the plunger in either the locked or unlocked (released) position. Correspondingly shaped barrel flange and finger grip are optionally provided for an exterior indicator of plunger condition as locked or unlocked. As stated above, barrel flange and finger grip can have corresponding or different shapes including those known to those of skill in the art.

Not shown in the figures is an aspect of syringe assembly that comprises a plunger having multiple structures of one or more of a teeth section and/or one or more of a flat section. As shown in the figures, for example FIG. 1, two structures, teeth section 121 and flat section 140, are formed into longitudinal shaft 124 of plunger 120 and each structure is located on adjacent portions of longitudinal shaft 124. In an aspect, a plunger comprises two teeth sections. For example, each of the two teeth sections are located apart from each other around the circumference of shaft 124, such as 180 degrees from each other, and are separated by a flat section. Like the teeth shown in the figures, the teeth are cut into the plunger shaft so that the teeth sections have the same radius as the plunger shaft and the diameter of the plunger shaft is maintained so that the plunger moves easily within the barrel lumen. The flat section separating the teeth structures is as described as above, and is cut into the plunger shaft and extends longitudinally along the plunger shaft for at least the length of teeth 121, and extends from a proximal edge to a distal edge. In an aspect, a syringe assembly comprises a plunger with a second flat section that is located 180 degrees from a first flat section. When the plunger is axially rotated around its longitudinal axis, so that the flat section faces the barrel key which is proximally located near the barrel proximal opening and within the barrel lumen, so that the flat section of the plunger faces and is adjacent to the barrel key, the plunger can move easily in a proximal or distal direction within the barrel and cannot be locked. This is the released position.

In an aspect, a syringe assembly disclosed herein comprises a second flat section that is located 180 degrees from a first flat section. In an aspect, a syringe assembly disclosed herein comprises two flat sections that are located 180 degrees from each other and are separated by a teeth structure, so that a plunger comprises two teeth structures and two flat sections. For example, in traversing axially around a plunger in a 360 degree rotation, one would find a teeth section, a flat section, a teeth section and a flat section. Locking a plunger comprises moving the plunger so that the barrel key is positioned between two teeth or a tooth and an edge, such as rotating the plunger in an axial position, for example in a 90 degree turn. Releasing the plunger from the locked position comprises moving the plunger axially so that one of the flat sections faces the barrel key of the syringe lumen by disengaging the barrel key from its position between two teeth or a tooth and an edge. As can be understood from the disclosure herein, a plunger may comprise one or more teeth structures, and one or more flat section structures. For example, a plunger may have two teeth structures and one flat section structure, two teeth structures and two flat section structures, one teeth structure and one flat section structure, one teeth structure and two flat section structures, or more than two or combinations thereof in different portions of a plunger.

Disclosed herein are methods of using a syringe assembly as disclosed herein. A method comprises some or all of the following steps, which are illustrated using FIG. 1 references. Plunger 120 is positioned longitudinally within lumen 111 at a position that is advantageous for either drawing material a material, such as a gas, liquid or gel, into, or pushing material out of lumen 111. Before or after positioning plunger 120 and moving material into or out of lumen 111, inlet/outlet port 114 is attached to an article. For example, inlet/outlet port 114 may be shaped to form a luer lock when mated with the corresponding shape on the article. An article can be a catheter, a balloon fitting, a needle, a container, or other known articles used in procedures for treating or diagnosing subjects. In an example, plunger 120 is moved in a proximal direction to draw air into lumen 111 and the plunger 120 is rotated so that two of teeth 121 bracket barrel key 118 and lock plunger 120 in that longitudinal position within lumen 111. Inlet/outlet port 114 is fluidly attached to a fitting of a catheter balloon. The catheter is positioned where desired in a subject and plunger 120 is unlocked (released) by rotating plunger 120 to move teeth 121 away from barrel key 118 and position flat section 140 adjacent to or to face barrel key 118. Once released/unlocked, plunger 120 is moved in a distal direction to move air from lumen 111 to the balloon lumen and inflate the balloon. Once the balloon is inflated to the desired volume by the movement of plunger 120 from one longitudinal position in lumen 111 to a more distal longitudinal position and pushed air from lumen 111, plunger 120 is locked in that longitudinal position by rotating plunger 120 so that flat section 140 no longer faces barrel key 118 and instead, barrel key 118 is now bracketed by two of teeth 121 or by distal edge 129 and tooth 121c.

The balloon can be inflated to any desired volume, from only a portion of the volume to the entire volume of the material contained by the syringe assembly (contained within lumen 111), and maintained at that desired volume, without further interaction by the operator, because syringe assembly 110 can be variably locked at any of the possible longitudinal positions. Syringe assembly 100 can provide all or just a portion of the air from its lumen to the balloon and then plunger 120 is locked at the longitudinal position reached when sufficient material has been expelled from the syringe lumen. The syringe assembly can maintain the longitudinal position of plunger 120, and thus the inflated balloon, for the desired time without interaction, stabilization or movement by the operator. This characteristic of being able to provide a desired and/or variable amount of material from a syringe assembly, and maintain that status without operator interaction, makes the disclosed variably lockable syringe assemblies different from syringe assemblies with no locking ability or only one locked position. A syringe assembly that does not lock can provide all or none of a material in in its lumen, and for any amounts between all or none, an operator must continue to grip and try to stabilize the plunger at the inbetween position and also respond to movement by the subject and resistant forces acting on the syringe assembly and the material. Additionally, to meet the needs of providing differing amounts of material to an article or a subject, operators have resorted to having available syringe assemblies of varying volumes and sizes so that varying amounts of material can be provided. A disclosed syringe assembly overcomes the need for having many differently sized syringe assemblies because one disclosed variably lockable syringe assembly can reliably and accurately provide variable amounts of material. In contrast, an unlockable syringe can provide varying amounts, but operator error and medical error is introduced by relying on the operator to maintain the unlockable syringe plunger at one location in an accurate and reliable manner.

When the balloon is to be deflated, plunger 120 is rotated within lumen 111 so that barrel key 118 is no longer bracketed by teeth or a tooth and an edge, and so that flat section 140 faces barrel key 118, i.e., plunger 120 is released or unlocked. In the unlocked position, plunger 120 is then moved in a proximal direction to draw the air within the balloon into lumen 111 and deflate the balloon.

A method for dispensing a material to a subject, comprises
a. positioning a locked variably lockable syringe assembly comprising a barrel having an inlet/outlet port, and a plunger, and comprising the material within the barrel of the syringe assembly, at or on the location of a subject receiving the material;
b. unlocking the variably lockable syringe assembly;
c. delivering a desired amount of the material by moving the plunger in a distal position to move at least a portion of the contained material from the syringe assembly out the inlet/outlet port and into or on the subject;
d. locking the variably lockable syringe assembly; and
e. removing the locked variably lockable syringe assembly from the subject.

Optionally, steps a) through e) can be repeated one or more times.

A method for providing material to an article, attaching a locked variably lockable syringe assembly comprising a barrel having an inlet/outlet port, and a plunger, and comprising the material within the barrel of the syringe assembly, to an article;
a. unlocking the variably lockable syringe assembly;
b. moving the plunger in a distal position to move at least a portion of the contained material from the syringe assembly out the inlet/outlet port;
c. locking the variably lockable syringe assembly;
d. optionally, unlocking the variably lockable syringe assembly and moving the plunger in a proximal direction to move at least a portion of the expelled material into the barrel via the inlet/outlet port; and
e. detaching the locked variably lockable syringe assembly from the article.

Materials that can be provided by a disclosed device comprise solutions, suspensions, liquids, gases, air, saline, pharmaceutical solutions comprising active agents, solutions comprising biological cells or cellular components, mixtures and combinations of these, and materials that can be dispensed by syringe assemblies that are known to those of skill in the art.

A system of the present disclosure comprises providing a device disclosed herein, or a device disclosed herein with a composition disclosed herein. A system disclosed herein comprises a device disclosed herein, and optionally, a composition disclosed herein. A system disclosed herein comprises a device disclosed herein having a plunger comprising at least one teeth structure and at least one flat section structure, and a syringe barrel comprising a barrel key, and optionally, a composition disclosed herein A system and/or a device disclosed herein may be packaged in a sterile container.

A kit of the present invention comprises a device disclosed herein which is contained in a kit container, and optionally directions for use of the device. A kit of the present disclosure comprises a container containing a device disclosed herein, with a composition disclosed herein, and optionally directions for use of the device. A kit disclosed herein comprises a container comprising a device disclosed herein, and optionally, a composition disclosed herein, with directions for using the device. A kit disclosed herein comprises a container comprising a device disclosed herein having a plunger comprising at least one teeth structure and at least one flat section structure, and a syringe barrel comprising a barrel key, and optionally, a composition disclosed herein with directions for using the device. A kit and/or a device disclosed herein may be packaged in a sterile container.

### Definitions

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, EIZ specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, when a compound is referred to as a monomer or a compound, it is understood that this is not interpreted as one molecule or one compound. For example, two monomers generally refers to two different monomers, and not two molecules.

As used herein, the terms “optional” or “optionally” means that the subsequently described event or circumstance can or cannot occur, and that the description includes instances where said event or circumstance occurs and instances where it does not.

As used herein, the terms “about,” “approximate,” and “at or about” mean that the amount or value in question can be the exact value designated or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In an aspect, a mammalian subject is a human. A patient refers to a subject afflicted with a disease or disorder or requiring contraception. The term “patient” includes human and veterinary subjects.

As used herein, the terms “administering” and “administration” refer to any method of providing a disclosed composition to a subject.

As used herein, the terms “comprises,” “comprising,” “includes,” “including,” “containing,” “characterized by,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus.

The transitional phrase “consisting of” excludes any element, step, or ingredient not specified in the claim, closing the claim to the inclusion of materials other than those recited except for impurities ordinarily associated therewith. When the phrase “consists of” appears in a clause of the body of a claim, rather than immediately following the preamble, it limits only the element set forth in that clause; other elements are not excluded from the claim as a whole.

The transitional phrase “consisting essentially of” limits the scope of a claim to the specified materials or steps and those that do not materially affect the basic and novel characteristic(s) of the claimed invention. A “consisting essentially of” claim occupies a middle ground between closed claims that are written in a “consisting of” format and fully open claims that are drafted in a “comprising” format. Optional additives as defined herein, at a level that is appropriate for such additives, and minor impurities are not excluded from a composition by the term “consisting essentially of”.

When a composition, a process, a structure, or a portion of a composition, a process, or a structure, is described herein using an open-ended term such as “comprising,” unless otherwise stated the description also includes an embodiment that “consists essentially of” or “consists of the elements of the composition, the process, the structure, or the portion of the composition, the process, or the structure.

The articles “a” and “an” may be employed in connection with various elements and components of compositions, processes or structures described herein. This is merely for convenience and to give a general sense of the compositions, processes or structures. Such a description includes “one or at least one” of the elements or components. Moreover, as used herein, the singular articles also include a description of a plurality of elements or components, unless it is apparent from a specific context that the plural is excluded.

The term “about” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such.

The term “or”, as used herein, is inclusive; that is, the phrase “A or B” means “A, B, or both A and B”. More specifically, a condition “A or B” is satisfied by any one of the following: A is true (or present) and Bis false (or not present); A is false (or not present) and Bis true (or present); or both A and B are true (or present). Exclusive “or” is designated herein by terms such as “either A or B” and “one of A or B”, for example.

In addition, the ranges set forth herein include their endpoints unless expressly stated otherwise. Further, when an amount, concentration, or other value or parameter is given as a range, one or more preferred ranges or a list of upper preferable values and lower preferable values, this is to be understood as specifically disclosing all ranges formed from any pair of any upper range limit or preferred value and any lower range limit or preferred value, regardless of whether such pairs are separately disclosed. The scope of the invention is not limited to the specific values recited when defining a range.

When materials, methods, or machinery are described herein with the term “known to those of skill in the art”, “conventional” or a synonymous word or phrase, the term signifies that materials, methods, and machinery that are conventional at the time of filing the present application are encompassed by this description. Also encompassed are materials, methods, and machinery that are not presently conventional, but that will have become recognized in the art as suitable for a similar purpose.

Unless stated otherwise, all percentages, parts, ratios, and like amounts, are defined by weight.

All patents, patent applications and references included herein are specifically incorporated by reference in their entireties.

It should be understood, of course, that the foregoing relates only to embodiments of the present disclosure and that numerous modifications or alterations may be made therein without departing from the spirit and the scope of the disclosure as set forth in this disclosure.

The present disclosure is further illustrated by the examples contained herein, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present disclosure and/or the scope of the appended claims.

Although the exemplary embodiments of the present invention describe in detail methods, syringe assembly devices, delivery systems, and compositions, the present invention is not limited to these embodiments. There are numerous modifications or alterations that may suggest themselves to those skilled in the art for use of the methods, delivery systems, and compositions herein for the diagnosis or treatment of a variety of conduits in both human and non-human mammals.

The present invention is further illustrated by way of the examples contained herein, which are provided for clarity of understanding. The exemplary embodiments should not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

1. A syringe assembly comprising:
a) a barrel comprising a barrel key located within the barrel lumen at a proximal end of the barrel;
b) a plunger comprising at least one teeth section structure and at least one flat section structure;
wherein the plunger is lockable along the lumen of the barrel between the distal end of the lumen and the proximal end of the lumen by positioning the barrel key between two teeth or between a tooth and an edge; and
wherein the plunger is unlockable by moving a flat section of the plunger to face or be adjacent to the barrel key.

2. The syringe assembly of claim 1, wherein the teeth section structure comprises multiple teeth

3. The syringe assembly of claim 1, wherein the teeth section structure comprises an edge structure.

4. The syringe assembly of claim 1, wherein the teeth section structure comprises multiple teeth and at least one edge structure.

5. The syringe assembly of claim 1, wherein the teeth section structure comprises multiple teeth and a proximal edge structure and a distal edge structure.

6. The syringe assembly of claim 1, wherein the plunger comprises two teeth section structures.

7. The syringe assembly of claim 1, wherein the barrel comprises indicia.

8. The syringe assembly of claim 1, wherein the barrel further comprises a distal inlet/outlet port.

9. The syringe assembly of claim 8, wherein an article is attached to the distal inlet/outlet port.

10. The syringe assembly of claim 9, wherein the article is a catheter, tubing, a needle or a medical device.

11. A method of inflating a catheter balloon, comprising
a) attaching a syringe assembly comprising i) a barrel comprising a fluid composition, having a proximal end and a distal end, comprising a barrel key located within the barrel lumen at a proximal end of the barrel, and an inlet/outlet port at the distal end; and ii) a plunger comprising at least one teeth section structure and at least one flat section structure; wherein the plunger is lockable along the lumen of the barrel between the distal end of the lumen and the proximal end of the lumen by positioning the barrel key between two teeth or between a tooth and an edge; and wherein the plunger is unlockable by moving a flat section of the plunger to face or be adjacent to the barrel key; to a port in fluid connection with a balloon lumen;
b) moving the plunger in a distal direction within the barrel lumen to move at least a portion of the fluid through and out the inlet/outlet port and into a balloon lumen; and optionally,
c) locking the plunger at a desired position within the barrel lumen by rotating the plunger so that the barrel key is positioned between two teeth or between a tooth and an edge.

12. The method of claim 11, wherein the port in fluid connection with the balloon lumen is connected to a catheter comprising the balloon.

13. The method of claim 11, wherein the port in fluid connection with the balloon lumen is connected to a medical device.

14. The method of claim 11, further comprising deflating the balloon by unlocking the plunger wherein the plunger is rotated so that the flat section structure is facing the barrel key.

15. The method of claim 14, further comprising moving the plunger in a proximal direction to reduce the volume of the inflated balloon.

16. The method of claim 11, wherein the fluid is air or a liquid.

17. The method of claim 11, wherein the teeth section structure of the syringe assembly comprises multiple teeth and at least one edge structure.

18. The method of claim 11, wherein the teeth section structure of the syringe assembly comprises multiple teeth and a proximal edge structure and a distal edge structure.

19. The method of claim 11, wherein the plunger of the syringe assembly comprises two teeth section structures.

20. A method for injecting a composition into a subject comprising, a) attaching a syringe assembly comprising i) a barrel containing a composition, having a proximal end and a distal end, and comprising a barrel key located within the barrel lumen at a proximal end of the barrel, and an inlet/outlet port at the distal end; and ii) a plunger comprising at least one teeth section structure and at least one flat section structure; wherein the plunger is lockable along the lumen of the barrel between the distal end of the lumen and the proximal end of the lumen by positioning the barrel key between two teeth or between a tooth and an edge; and wherein the plunger is unlockable by moving a flat section of the plunger to face or be adjacent to the barrel key; to a needle useful for injection;
a) inserting the needle into a subject at a target site;
b) moving the plunger a desired distance in a distal direction within the barrel lumen to move at least a portion of the composition through and out the inlet/outlet port, through the needle and into the target site to inject a desired amount of the composition,
c) locking the plunger at the desired distance position within the barrel lumen by rotating the plunger so that the barrel key is positioned between two teeth or between a tooth and an edge;
d) withdrawing the needle from the target site, and optionally, repeating steps a) to d) at a second target site.

21. The method of claim 12, further comprising deflating the balloon by unlocking the plunger wherein the plunger is rotated so that the flat section structure is facing the barrel key

22. The method of claim 13, further comprising deflating the balloon by unlocking the plunger wherein the plunger is rotated so that the flat section structure is facing the barrel key.
